(19) United States
(12) Patent Application Publication
(10) Pub. No.: US 2019/0392932 A1
(43) Pub. Date: Dec. 26, 2019

(54) USE OF NEAR FIELD COMMUNICATIONS TECHNOLOGY TO TRANSFER PATIENT RELATED DATA TO DIAGNOSTIC MEDICAL EQUIPMENT IN A MEDICAL OFFICE OR OTHER HEALTHCARE FACILITY

(71) Applicant: David A. Wallace, Los Angeles, CA (US)

(72) Inventor: David A. Wallace, Los Angeles, CA (US)

(21) Appl. No.: 16/447,642

(22) Filed: Jun. 20, 2019

Related U.S. Application Data
(60) Provisional application No. 62/687,868, filed on Jun. 21, 2018.

(51) Int. Cl. G16H 10/65; G16H 80/00; A61B 5/00

(52) U.S. Cl. CPC G16H 10/65; G16H 80/00; H04L 67/12; A61B 5/0022; A61B 5/7465

(57) ABSTRACT

A method for transferring patient-related data stored on a smartphone to diagnostic medical equipment utilizing near-field communications (NFC) includes downloading a protected health information (PHI) transfer software application onto the smartphone; initiating search for NFC-capable devices in adjacent areas to the smartphone; establishing a communication session between the smartphone and any found NFC-capable device; initiating data transfer protocol and transferring patient's PHI from smartphone to the found NFC-capable device; and closing the communication session and the PHI transfer software application. The method further includes verifying accuracy of a patient's PHI before initiating the data transfer protocol.

### RELATED APPLICATIONS

This application claims priority to U.S. provisional patent application Ser. No. 62/687,868, filed Jun. 21, 2018, entitled “Use Of Near Field Communications Technology To Transfer Patient-Related Data To Diagnostic Medical Equipment In A Medical Office Or Other Healthcare Facility,” the disclosure of which is hereby incorporated by reference.

### BACKGROUND

When patients come to a doctor's office or healthcare facility, diagnostic procedures are typically performed as a part of a routine evaluation. For example, in the office of an internist or general practitioner, testing may include recording of vital signs (temperature, pulse, blood pressure), body weight, EKG, routine blood tests and other diagnostic studies. In the offices of eye care practitioners, patients may have specialized diagnostic testing performed including autorefraction, corneal topography, retinal imaging, axial biometry, and/or optical coherence tomography, etc.

Patient information must be entered into these diagnostic devices and/or a computing device attached thereto, so that the diagnostic test results and/or images are assigned or associated with the correct patient. Currently, such patient information is entered utilizing manual data entry methods. Manual data entry is problematic due to risk of typographic errors, duplicate entries, incorrect data entry, misspelling of names or vital personal information, and/or complete lack of entering certain fields for any number of reasons.

Thus, a need exists for easy, quick and accurate transfer of patient's personal information into any of a multiplicity of diagnostic devices immediately prior to testing.

### BRIEF DESCRIPTION OF DRAWINGS

The patent or application file contains at least one drawing.

A better understanding of the features, advantages and principles of the present disclosure will be obtained by reference to the following detailed description that sets forth illustrative embodiments, and the accompanying drawings of which are described below.

FIG. 1 illustrates a system where protected health information (“PHI”) is transferred between a mobile communications device and medical diagnostic equipment according to some embodiments;

FIG. 2 illustrates a method of utilizing NEC communications in a medical diagnostic environment according to some embodiments; and

FIG. 3 illustrates a mobile communications device or computing device including an NFC communications module according to some embodiments.

### DETAILED DESCRIPTION OF THE INVENTION

The following detailed description and provides a better understanding of the features and advantages of the subject matter described in the present disclosure in accordance with the embodiments disclosed herein. Although the detailed description includes many specific embodiments, these are provided by way of example only and should not be construed as limiting the scope of the subject matter disclosed herein.

The subject matter claimed herein relates to communicating or transferring certain identifying information typically classified as protected health information (“PHI”) from a mobile communications device to computerized diagnostic equipment in a medical provider's facility (and/or in other commercial facilities). Although most discussion is focused on use with medical diagnostic devices, the methods and devices described herein apply to other transfers of protected and/or confidential information from a mobile communications device to another computing device in commercial and/or educational settings (e.g., entry into buildings, authentication of individuals at facilities, etc.). Near-Field communications (NFC) (e.g., via a NFC chipset and antenna or NFC communications module) may be utilized to transfer a variety of information (including PHI) from one device to another, or from an identifier tag associated with a subject (similar to an RFID tag) to an NFC-enabled device attached to or integrated within medical diagnostic testing instrument or device. In some embodiments, the NFC communications module typically may consist of an NFC-appropriate antenna and an NFC chipset (which may be referred to as an NFC transceiver) for facilitating recognition of another NFC-enabled device, and effecting data transfer between the two devices. In some embodiments, the NFC communication module may include some functionality incorporated within software and/or some functionality incorporated within hardware. Alternatively, one NFC-enabled device may be able to read an NFC-enabled tag (analogous to an RFID tag) issued by the medical provider's office and associated with a subject.

A consortium of manufacturers including Sony, Nokia and Ericson developed hardware and software protocols for NFC in about 2002. Early usage was rather limited as the proliferation of mobile communication devices (e.g., smartphones) had not yet reached significant proportions. It is anticipated that by the year 2020, something in excess of 80% of the world population will own or have access to a mobile communications device. With this extremely widespread proliferation, it becomes feasible to consider use of a patient's own mobile device to warehouse PHI and selectively convey the patient's PHI to testing or diagnostic devices within a medical provider's office that they may visit in order to receive medical care. Such a PHI transfer system would only be possible if the patient's mobile communications device and the medical diagnostic equipment (or a computing device associated with the medical diagnostic equipment) both include NFC communication modules (e.g., circuitry and antennas as well as software (e.g., computer-readable instructions executable by one or more processors or controllers)) to allow communication, handshake, data transfer and verification of completion of the PHI transfer process. In some embodiments, it is only smart phones or mobile phones that included NFC communication modules and some other mobile communication devices do not include NFC communication modules.

Near-Field Communications (NFC) allows devices to communicate data and/or information to and/or from each other when the devices are in near proximity to each other. NFC operates at 13.56 MHz on an ISO/IEC 18000-3 air interface. NFC operates at data frequency rates ranging from 106 kbit/s to 424 kbit/s. NFC involves an initiator and a target, where the initiator actively generates an RF field which may power a passive target. By having passive targets, NFC targets may have very simple form factors such as unpowered tags, stickers, key fobs, or cards. In alternative embodiments, NFC may have an active target and the target may communicate with an initiator NFC device. NFC on its own does not provide a secure channel but software and/or combined software/hardware applications may utilize a higher-layer cryptographic protocol to establish a secure channel(s) of communication. In some embodiments, many mobile communication devices include one or more NFC communication modules (e.g., NFC antennas and/or chipsets (referred to hereinafter as an “NFC transceiver”), or more generally as “NFC systems”) to allow communications with other mobile communications devices or computing devices including NFC communication modules. Typical proximity between NFC-enabled devices needs to be about 4″ (10 cm) or less to enable recognition, linkage and data transfer. A modern public application of NFC transfer including encrypted data is Apple Pay, enabling (encrypted) conveyance of credit card information from a smartphone to an NFC-enabled point-of-sale processing terminal.

In a medical diagnostic environment, a scenario exists in which it may often be desirable to wirelessly transfer patient's encrypted PHI to diagnostic devices immediately prior to diagnostic examinations. This functionality and feature does not currently does not exist and is the subject matter of the present application.

In the modern era, most patients have their own mobile communication devices, which may be a smartphone. While the specification refers to mobile communication devices (or smartphones), a mobile communication device may also be a tablet computing device, a small laptop device and/or a wearable computing device (smart glasses (e.g., Google Glasses) or smart watches). In some embodiments, this mobile communication device can be utilized for storing and transferring certain select patient PHI. In some embodiments, a patient's PHI may include first name, last name, middle initial, date of birth, gender, medical record number, email address, and/or possibly other identifiers. In some embodiments, a patient's PHI may include a social security number or other personal identifier. In some embodiments, a patient's PHI may include additional medical-related parameters. In some embodiments, a patient's insurance information is not included in PHI because it is not needed and may not be classified as protected health information. In some embodiments, a patient's financial information may not be included in PHI because it is not classified as protected health information. In some embodiments, the patient's PHI may be stored in a secure area of one or more memory devices of the mobile communication device. In some embodiments, the patient's PHI may be protected by encryption, secure keys and/or passwords. In some embodiments, the mobile communications device may also include computer-readable instructions stored in one or more memory device that are executable by a processor to initiate and perform the patient PHI transfer or communication process. This may be referred to as Patient PHI transfer software or medical PHI transfer software.

In some embodiments, a medical provider or a medical provider facility may provide a software application to patient's that allows patients to make appointments, communicate messages to medical providers who are their primary care physician, review test results, input and/or review prescribed medications, make appointments and/or pay bills. In some embodiments, this software may be referred to as Medical Provider software, Patient Medical software, or a Medical Provider software application. In some embodiments, some or all of the Medical Provider software application may be downloaded onto a patient's mobile communication device. In some embodiments, the Patient PHI transfer software may be part of or integrated within the Medical Provider software. In some embodiments, the Medical Provider software may request the user's PHI and may store the user's PHI in one or more memory devices of the mobile communication device during an initial usage of the Medical Provider software or during registration of the Medical Provider software. In some embodiments, because it is protected health information (PHI), the patient may need to agree to and/or consent to (electronically) the storage of the PHI in the PHI transfer software and/or the Medical Provider software. In some embodiments, the Medical Provider software may store the entered PHI data in a secure memory portion of the one or more memory devices of the mobile communications device to prevent access to the patient's PHI data without a proper password and/or following of an authentication process (and to protect the PHI data from malware or other malicious software applications.

In some embodiments, the medical diagnostic equipment may need to receive patient PHI in order to either 1) create a new record or 2) search for or locate an existing record, and then may associate the diagnostic testing with the new record and/or existing record. In some embodiments, the medical diagnostic equipment may generate records including 1) the generated diagnostic parameters, measurements and/or data; and/or 2) a portion or all of the patient's PHI. In some embodiments, the medical diagnostic equipment (or a computing device associated with the medical diagnostic equipment or an NFC-enabled accessory device) may communicate the generated records to other medical computing devices and/or to a medical record repository.

In some embodiments, a patient's PHI may need to be protected from third-party interception. Thus, if devices are transferring this information, the patient's PHI needs to be communicated between devices (e.g., a mobile communication device and/or medical diagnostic equipment) via specific frequencies and/or normally via encrypted channels. In some embodiments, the use of NFC communication modules or NFC circuitry and antennas (along with software that appropriately encrypts the information) may readily provide this functionality.

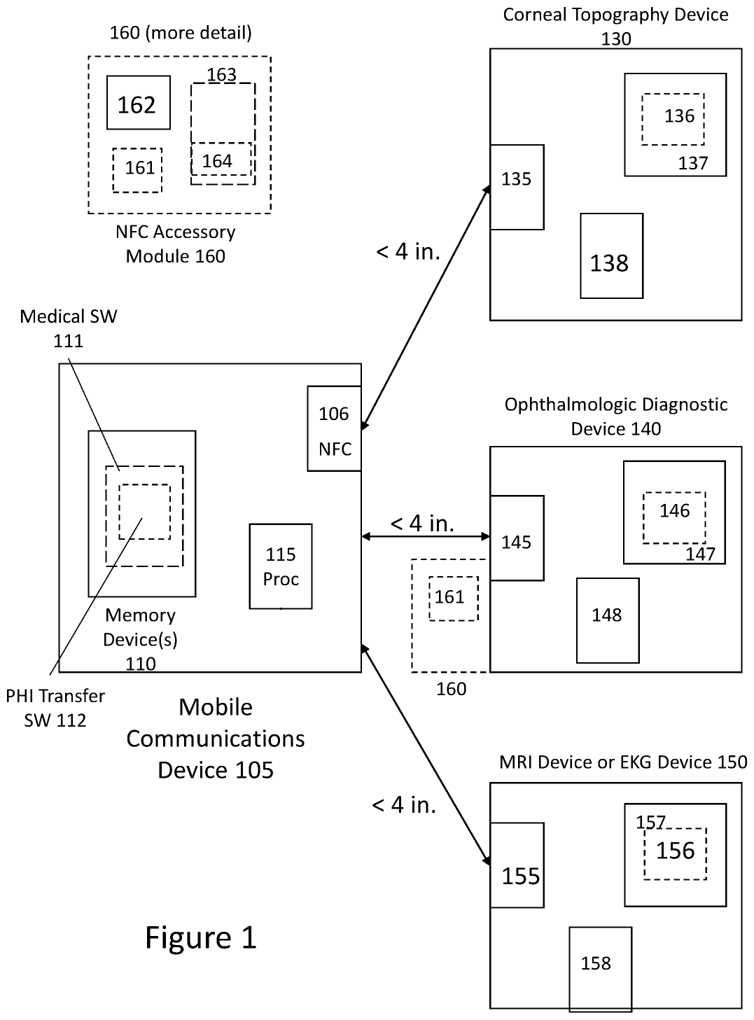

FIG. 1 illustrates a system where protected health information (“PHI”) is transferred between a mobile communications device and medical diagnostic equipment according to some embodiments. In some embodiments, the system may comprise a mobile communication device 105, a corneal topography device 130 and/or an ophthalmologic diagnostic device 140. In some embodiments, in order for the NFC communications to be enabled and data transfer to work, the mobile communication device 105 should be within 4 inches (or 10 centimeters) or less of the corneal topography device 130 and/or the opthalmological diagnostic device 140. In some embodiments, the mobile communication device 130 may comprise one or more processors 115, one or more memory devices 110, and/or one or more NFC communications modules 106. In some embodiments, in the mobile communication device 105, computer-readable instructions executable by one or more processors 115 may comprise the Medical Provider software 111. In some embodiments, the PHI transfer software 112 may be a part of or integrated within the Medical Provider software 111. In some embodiments, when a patient or user downloads the Medical Provider software 111, the PHI transfer software 112 may also be downloaded into the one or more memory devices 110 of the mobile communication device 105. In some embodiments, the PHI transfer software 112 may be stored in a secure portion of the one or more memory devices 110. In some embodiments, only the patient PHI data may be stored in the secure portion of the one or more memory devices 110 in order to prevent other applications from accessing the patient PHI data.

In some embodiments, a system may include a corneal topography device 130 and/or another ophthalmologic diagnostic device 140. In some embodiments, a system may include another medical diagnostic device 150, such as a Magnetic Resonance Imaging (MRI) diagnostic device and/or an electrocardiogram (EKG) device. In some embodiments, a patient may walk into an ophthalmologist's office and may have an examination scheduled on a corneal topography device 130. In some embodiments, the corneal topography device 130 may comprise a NFC communications module 135, one or more processors 138, one or more memory devices 137 and/or computer-readable instructions 136 (including, but not limited to computer-readable instructions directed to data transfer functionality via the NFC communications module 135). In some embodiments, the computer-readable instructions 146 executable by the one or more processors 148 may allow the corneal topography device 130 to engage in data transfer sessions and/or communications with the mobile communication device 105 via the NFC communications module 135 of the corneal topography device 130. In some embodiments, when a patient comes into a room with the corneal topography device 130, the patient's mobile communication device (e.g., specifically the PHI transfer software) may transfer the patient's PHI data to the corneal topography device 130 (or a computing device associated with the corneal topography device 130)

In some embodiments, the ophthalmologic diagnostic device 140 may be a autorefraction, optical biometry, optical coherence tomography, specular microscopy, and digital camera systems for retinal and/or slit-lamp microscope photography, and/or other similar ophthalmologic diagnostic devices. In some embodiments, the ophthalmologic diagnostic device 140 may comprise an NFC communications module 145, one or more processors 148, one or more memory devices 147 and/or computer-readable instructions 146 stored in the one or more memory devices and executable by the one or more processors 148. In some embodiments, the computer-readable instructions 146 executable by the one or more processors 148 may allow the ophthalmologic diagnostic device 140 to engage in data transfer sessions and/or communications with the mobile communication device 105 via the NFC communications module 145 of the ophthalmologic diagnostic device 140. In some embodiments, when a patient comes into a room with the ophthalmologic diagnostic device 140, the patient's mobile communication device (e.g., specifically the PHI transfer software) may transfer the patient's PHI data to the ophthalmologic diagnostic device 140 (or a computing device associated with the ophthalmologic diagnostic device 140).

In some embodiments, the medical diagnostic device (e.g., MRI or EKG device) 150 may comprise an NFC communications module 155, one or more processors 158, one or more memory devices 157 and/or computer-readable instructions 156 stored in the one or more memory devices 157. In some embodiments, memory devices and executable by the one or more processors 148. In some embodiments, the computer-readable instructions 156 executable by the one or more processors 158 may allow the medical diagnostic device 150 to engage in data transfer sessions and/or communications with the mobile communication device 105 via the NFC communications module 155 of the medical diagnostic device 150. In some embodiments, when a patient comes into a room with the medical diagnostic device 150, the patient's mobile communication device (e.g., specifically the PHI transfer software) may transfer the patient's PHI data to the medical diagnostic device 150 (or a computing device associated with the medical diagnostic device 150).

In some embodiments, any of the NFC communication modules 106, 135, 145, and 155 may comprise one or more NFC chipsets and antennas (e.g., an NFC transceiver). In alternative embodiments, an accessory device associated with, coupled to, connected to and/or attached to the mobile communication device 105, the diagnostic medical equipment (e.g., the corneal topography device 130, the opthalmological diagnostic device 140 and/or the MRI or EKG device 150) may include one or more NFC communication modules 161 (e.g., the one or more NFC chipsets and antennas and/or associated computer-readable instructions executable by one or more processors or controllers). In other words, these accessory devices 160 may be attached to any of the medical diagnostic devices listed above and may include an NFC communication module 161, one or more processors 162, one or more memory devices 163 and/or computer-readable instructions 164 executable by the one or more processors to perform communications via the NFC protocol with other devices (as is shown in FIG. 1). In some embodiments, these NFC accessory devices 160 may be purchased separately from the medical diagnostic devices and may connect or coupled to available interfaces of the diagnostic devices. This provides an advantage of being able to make any of the existing diagnostic devices in the marketplace or in medical facilities (e.g., existing corneal topography devices 130, existing opthalmological diagnostic devices 140 and/or existing medical diagnostic devices 150 (e.g., MRI or EKG device) NFC-capable by adding or coupling an NFC communication module to the existing medical diagnostic devices. This reduces the expense of being able to add in this functionality.

In some embodiments, when a patient (and thus the patient's mobile communication device 105) enters into a facility, the patient may initiate the PHI transfer software to determine if any NFC-enabled devices are present in the area around the mobile communication device 105. This is one alternative. In alternative embodiments, because the patient has previously loaded the Medical Provider software, the patient may know that the medical facility has diagnostic devices that are NFC-enabled. Thus, in some embodiments, if a patient is scheduled for a diagnostic exam, the patient may move the mobile communication device 105 to within 4 inches (or 10 cm) of the diagnostic device (e.g., the corneal topography device 130, opthalmological diagnostic device 140 and/or the medical diagnostic device 150) and the computer-readable instructions 112 executable by the one or more processors 115 of the mobile communication device 105 may establish a communication path or communication session with the medical diagnostic device where the patient is being examined. In some embodiments, the computer-readable instructions 112 executable by the one or more processors 115 of the mobile communications device may transfer the patient's PHI data through the NFC communication module 106 to the associated NFC communication module (e.g., 135, 145, 155, or 161) of the medical diagnostic device in the room or facility where the patient is having the examination. In some embodiments, the medical diagnostic device may receive the patient's transferred PHI data and utilize the PHI data to link to the results (or be associated with the results) of the diagnostic test being performed on the patient. In some embodiments, the computer-readable instructions executable by the one or more processors of the medical diagnostic device may then communicate with the computer-readable instructions on the mobile communication device 105 to disconnect the NFC communication path or terminate the NFC communication sessions between the devices. This system and procedure provides the advantage of limiting the amount of mistakes that are made in manual data entry when the patient comes into an medical office or facility and provides protected personal information to the medical professional. In some embodiments, the use of NFC communications module allows an overall time for performing the diagnostic testing to be reduced because very little time is utilized to transfer the PHI data to the medical diagnostic device (e.g., corneal topography device 130, opthalmological diagnostic device 140 and/or the medical diagnostic device 150). In some embodiments, a display on the diagnostic equipment (or a display on the computing device associated with the medical diagnostic equipment) may display the patient PHI to allow the patient to view the transferred PHI data and verify the transferred PHI data is accurate.

In some embodiments, for example, the medical diagnostic equipment may be mobile communication device-based. In such embodiments, a patient's own mobile communication device may include a PHI transfer software app to transmit the patient PHI to the mobile communication device-based diagnostic device. In some embodiments, the mobile communication device-based diagnostic device may run, initiate and/or execute a software application enabling patient PHI transfer. In some embodiments, the PHI transfer software app may be clean, simple, elegant; and may communicate via encrypted communications channels at specific frequencies utilized by NFC transceivers and antennas (or the NFC communications module). This provides the advantage of eliminating incorrect data entry and potential duplicate entry that occurs with any manual data entry system. It is also a quick and seamless procedure for both the patient and the medical provider.

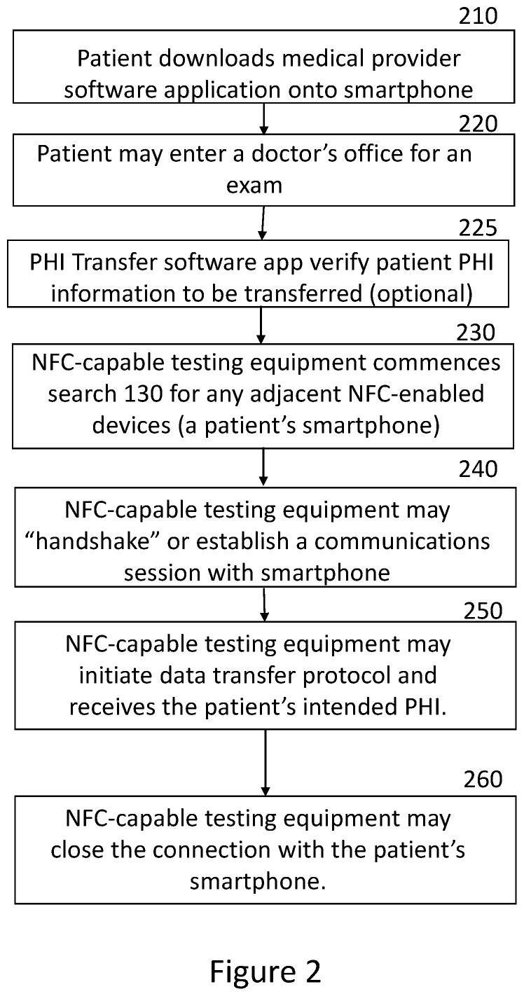

FIG. 2 illustrates a method of utilizing NFC communications in a medical diagnostic environment according to some embodiments. Below is a sample scenario of use and a flowchart of the patient PHI transfer application. This refers to a patient visiting a doctor's office. However, the same discussion may apply to patients visiting other medical facilities such as imaging centers, outpatient surgery centers and/or hospitals. In addition, this flowchart may be utilized with active and/or passive NFC devices. In addition, this flowchart may also be representative of an individual sharing protected information in other commercial settings, e.g., like a public library, a bank, a school or other places where personal information needs to be protected, but also utilized in order to verify identify and/or gain access and also applies to these other commercial settings.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed. The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein. In addition, while the process is described in a view or orientation where the medical diagnostic device discovers NFC-enabled mobile communications devices and/or initiates the communication session, the flowchart also applies to situations where the mobile communications device discovers the NFC-enabled medical diagnostic device and/or initiates the communication session with the NFC-enabled medical diagnostic device.

In some embodiments, a patient may download and/or install (210) Medical Provider software on their mobile communication device and the Medical Provider software may include PHI transfer software. In some embodiments, the PHI transfer software application may be part of a larger Medical Provider software application that includes other functionality as well, including warehousing information about appointments with provider offices, medication and dosage information, patient-doctor email aftercare instructions, and a variety of other patient health-relevant information. In some embodiments, the Medical Provider software may request that the patient enter protected health information (PHI) into the Medical Provider software. In some embodiments, the patient may be requested to verify and/or consent to storing the patient's PHI on the patient's mobile communication device utilizing the Medical Provider software. In some embodiments, for example, the patient may be presented with an electronic end-user license agreement (EULA) that the patient may need to accept and consent to in order to store, keep and/or utilize the patient's PHI. In some embodiments, the patient may typically have significant personal information stored on their personal mobile communication device. In some embodiments, the personal information may be referred to as protected health information (PHI). This PHI may include a patient's first name, last name, middle initial, date of birth, gender, email address, and other information such as home address, office address, etc. In some embodiments, the PHI may include a personal identifier (e.g., a social security number, a driver's license number, or another unique individual identifier). In some embodiments, the personal mobile communications/computing device may have an NFC chipset and antenna (e.g., a transceiver) and/or a NFC communications module. In some embodiments, the PHI transfer software application may interface with medical diagnostic equipment (e.g., an opthalmological diagnostic device or other medical diagnostic devices) and/or transferring select PHI from the mobile communication device to the medical diagnostic equipment. In some embodiments, a patient may have already downloaded the PHI transfer software application onto their mobile communications device in advance of visiting a particular provider's office.

In some embodiments, a patient may enter a doctor's office for an exam (220). In some embodiments, the PHI Transfer software application may ask the patient to either enter or verify certain personal information that will be transferred to any medical diagnostic device (225). In some embodiments, this may be an optional step. In some embodiments, this verification may be done via an input screen on a mobile communication device or via a voice response identifying that the correct personal information (e.g., PHI data) is going to be transferred. In some embodiments, such information might reasonably include first name, middle initial or full middle name, last name, date of birth, and other identifiers appropriate to particular provider offices or facilities including, for example, medical record number, provider office identifier, etc. In some embodiments, the entire complement of PHI data to be transferred (or a portion of the PHI data to be transferred) would be displayed on the patient's mobile communication device in a window on a display device of the mobile communication device, and authorization or confirmation would be obtained by clicking an appropriate button, a voice command, recognizing a gesture via image recognition and/or similar data input method. Once PHI data is entered or retrieved, and confirmed by the patient, the PHI transfer software application may be in a state ready to transfer the selected PHI data to a target medical diagnostic device via a NFC communication module.

In an ophthalmologist's office, for example, the medical diagnostic equipment can include corneal topography, autorefraction, optical biometry, optical coherence tomography, specular microscopy, and digital camera systems for retinal and/or slit-lamp microscope photography. In an internist's or a general practitioner's office, the medical diagnostic equipment may include an EKG, a blood pressure and heart rate monitor, X-ray machine and/or medical ultrasound diagnostic machine. In a hospital, the medical diagnostic equipment may include X-ray imaging, magnetic resonance imaging (“MRI”), computerized axial tomographic imaging (“CAT” scan), and/or diagnostic equipment that performs cardiac catheterization studies, pulmonary function studies, medical diagnostic ultrasound evaluations (echocardiogram, abdominal ultrasound, in-utero sonogram, etc.), blood tests and many others. The claimed subject matter, as mentioned before, may be utilized in other medical facilities and in combination with other medical diagnostic equipment. As discussed previously, in some embodiments, the manufacturer of such medical diagnostic devices may incorporate and/or integrate NFC communication modules (e.g., NFC chipsets and/or antennas) into the medical diagnostic devices. In some embodiments, the NFC communication module may be an add on and/or an accessory to the medical diagnostic device and may be connected, coupled, attached and/or adhered to the medical diagnostic device and then communicatively coupled and/or connected to the medical diagnostic device in order to transfer any information and/or data received from the NFC communication module. For example, in some embodiments, medical diagnostic equipment may have an integrated NFC communication module (chipset, antenna (e.g., transceiver) and operational firmware or software), or such NFC communication module may be attached to certain medical diagnostic equipment in a fashion analogous to how an Apple-Pay-capable point-of-sale transaction terminal is attached to a cash register.

In some embodiments, the NFC-capable medical diagnostic equipment (or computing device associated therewith) may have computer-readable instructions executable by one or more processors to commence search 230 for any adjacent NFC-enabled devices and/or systems, such as a patient's mobile communications device (or smartphone). In some embodiments, the NFC-capable medical diagnostic equipment may “handshake” or establish a communications session 240 with the patient mobile communication device. In some embodiments, the patient mobile communication device may handshake or establish a communications session with the NFC-capable medical diagnostic equipment or device, rather than having the NFC-capable medical diagnostic device initiate the communication session. In some embodiments, the patient mobile communication device screen may display that medical diagnostic equipment has established a secure communications session with the mobile communication device. In some embodiments, the NFC-capable diagnostic equipment may initiate data transfer protocol via the PHI data transfer application and receive the patient's intended PHI (250). In some embodiments, the patient's mobile communication device may initiate data transfer protocol via the PHI data transfer software application to communicate the patient's PHI to the NFC-capable diagnostic equipment. In embodiments, NFC-capable medical diagnostic equipment may then then close the connection (or terminate the communication session) with the patient's mobile communication device (260). In some embodiments, the patient's mobile communication device may terminate the communication session with the medical diagnostic equipment after completion of the PHI data transfer. Although the above-described process may be identified in terms of the medical diagnostic device initiating the different steps, the patient mobile communication device may initiate some or all of the different steps of the above-described process of FIG. 2.

From the perspective of the patient and his or her mobile communication device, the experience would seem straightforward. In some embodiments, when a patient enters any room where examination equipment is present, the patient would be asked to launch a specific application on their mobile communication device (e.g., a smartphone), then when the medical diagnostic testing equipment is ready, hold their mobile communication device (e.g., smartphone) in close proximity to the point of ideal proximity (NFC communication module and specifically NFC antenna) (e.g., less than 4 inches or 10 cm) on the medical diagnostic device. Once the two NFC-enabled devices (e.g., the mobile communication device and the medical diagnostic device) recognize they are in range of each other, the PHI software application on the patient's mobile communication device may initiate a communication session with the medical diagnostic equipment (or computing device associated with the medical diagnostic equipment) to transfer the patient's selected PHI to the medical diagnostic equipment (or the computer device associated with the medical diagnostic equipment). In some embodiments, the medical diagnostic equipment (or computing device) may initiate the communication session. In some embodiments, the PHI data transfer may be designed to be encrypted and authenticated. In some embodiments, the medical diagnostic equipment may be programmed to populate the appropriate patient data fields in the medical diagnostic equipment software with the patient's PHI data that was previously stored on the mobile communication device (e.g., smartphone) and then transferred to the medical diagnostic equipment. An advantage of this PHI data transfer process is that the process may occur almost instantaneously, seamlessly, automatically and/or without potential for data entry error associated with human data entry. In some embodiments, the technician or provider user can verify that the received information is accurate by reviewing the information received through the PHI transfer application on a display of the medical diagnostic equipment (or the computing device associated with the medical diagnostic equipment). In some embodiments, after the patient's mobile communication device has transferred or communicated the patient's PHI to the diagnostic equipment, the mobile communication device may close or deactivate the PHI transfer software application.

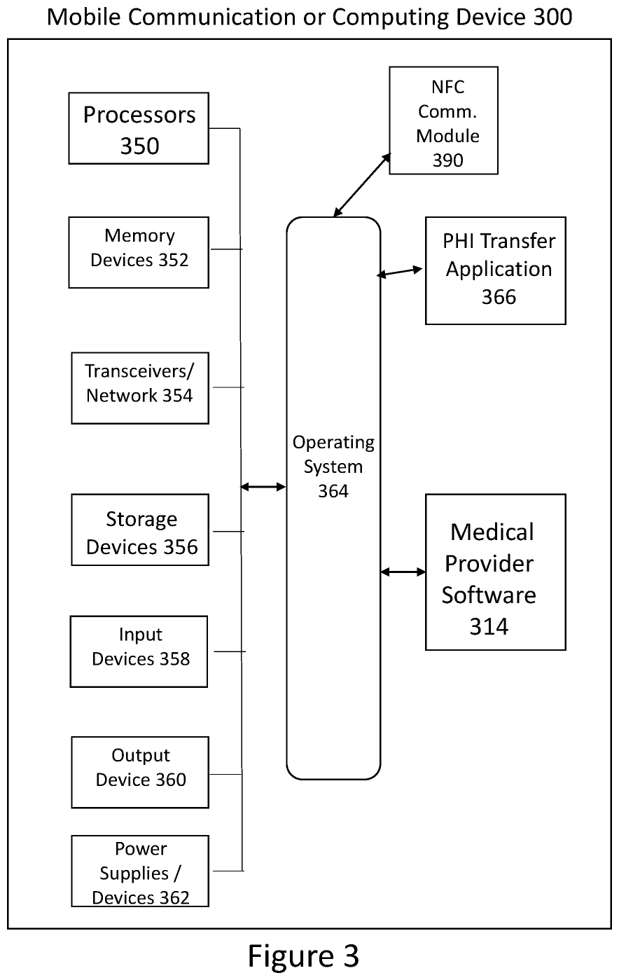

FIG. 3 illustrates a mobile communications device or computing device including an NFC communications module according to embodiments. As shown in the specific example of FIG. 3, in some embodiments, mobile computing device 300 includes one or more processors 350, one or more memory devices 352, one or more transceiver or communication network interfaces 354, one or more storage devices 356, one or more input devices 358, one or more output devices 360, and one or more power sources 362. In some embodiments, mobile communications device or computing device 300 may also include one or more operating systems 364 that are executable by one or more processors of the mobile communications device or computing device 300. In some embodiments, a mobile communications device or computing device 300, in one example, may further include a Medical Provider software 312 and one or more application software including but not limited to PHI transfer software 366 that are also executable by mobile communications device or computing device 300. Each of components 350, 352, 354, 356, 358, 360, 362, 364, 366, and 312 may be interconnected (physically, communicatively, or operatively) for inter-component communications. In some embodiments, the mobile communications device or computing device may comprise an NFC communications module 390 to communicate with other medical diagnostic devices and/or transfer PHI between the mobile communications device and the medical diagnostic devices. In some embodiments, the mobile communication devices such as smart phones or mobile phones may have NFC communication modules 390 incorporated within the device whereas other mobile communication devices may not. In some embodiments, the mobile communication devices that do not have an NFC communication module incorporated or integrated therein, may have an NFC accessory module attached, coupled and/or connected thereto. This may include older tablets and/or laptop computing devices. In some embodiments, all mobile communication devices may have NFC communication module 390 incorporated and/or integrated therein.

In some embodiments, one or more processors 350, in one example, are configured to at least implement functionality or process instructions for execution within the mobile communication device. For example, in some embodiments, one or more processors 350 may be capable of processing instructions stored in one or more memory devices 352 or instructions stored on one or more storage devices 356.

In embodiments, one or more memory devices 352, in one example, are configured to store information within mobile communications device or computing device 300 during operation. One or more memory devices 352, in some examples, is described as a computer-readable storage medium. In some examples, one or more memory devices 352 may be used as a temporary memory, although one or more memory devices 352 may be used as long-term storage. In embodiments, one or more memory devices 352, in some examples, is described as a volatile memory, meaning that one or more memory devices 352 may not maintain stored contents when mobile communications device or computing device 300 is powered off. Examples of volatile memories include random access memories (RAM), dynamic random access memories (DRAM), static random access memories (SRAM), and other forms of volatile memories known in the art. In some examples, one or more memory device 352 is used to store program instructions for execution by one or more processors 350. In embodiments, one or more memory devices 352, in one example, is used by software or applications running on mobile communications device or computing device 300 (e.g., patient medical provider software application 314 and/or other applications (e.g., the PHI data transfer software application 366) to temporarily store information during program execution.

In some embodiments, storage devices 356, in some examples, may also include one or more computer-readable storage media. In embodiments, storage devices 356 may be configured to store large amounts of information. In embodiments, storage devices 356 may further be configured for long-term storage of information. In some examples, storage devices 356 include non-volatile storage elements. Examples of such non-volatile storage elements include magnetic hard discs, optical discs, floppy discs, flash memories, or forms of electrically programmable memories (EPROM) or electrically erasable and programmable (EEPROM) memories. For the purposes of this disclosure one or memory devices or computer-readable storage mediums store computer-readable instructions, the computer-readable instructions may include computer program code that is executable by one or more processors. In embodiments, for example, and not being read as limiting, computer readable mediums may comprise computer readable storage media, for tangible or fixed storage of data, or communication media for transient interpretation of code-containing signals.

Mobile communications device or computing devices 300, in some examples, also includes one or more transceiver or communication network interfaces 354. Mobile communications device or computing device 300 in one example, utilizes transceiver or communication network interfaces 354 to communicate with external devices via one or more networks, such as one or more wireless communication networks. One or more transceiver or network interfaces 354 may be a network interface card, such as an Ethernet card, an optical transceiver, a radio frequency transceiver, or any other type of device that can send and receive information. Other examples of such network interfaces and/or transceivers may include personal area network (PAN), Bluetooth, 3G, 4G, 5G and WiFi radios in mobile computing devices as well as USB. In some examples, mobile communications device or computing device 300 utilizes one or more communication and network interfaces 354 to wirelessly communicate with an external device such as a server device or server computing device, a mobile phone, or other networked computing devices.

In some embodiments, a mobile communication or communications device, or computing device may comprise an NFC communication module 390. In some embodiments, the NFC communication module 390 (along with computer-readable instructions integrated within the NFC communication module and/or computer-readable instructions stored in the one or more memory devices 352 or storage devices 356) may allow the mobile communication device or computing device 300 to communicate with other NFC-enabled mobile communication devices, computing devices and/or medical diagnostic equipment utilizing the NFC protocol. In some embodiments, the NFC communication module 390 may be physically located external to the mobile communication device (or computing device or medical diagnostic device) 300 (e.g., it may be attached and/or coupled to the mobile communication device, computing device or medical diagnostic devices, as discussed above).

Mobile communications device or computing device 300, in one example, may also include one or more input devices 358. Input devices 358, in some examples, are configured to receive input from a user through tactile, audio, or video input. Examples of input devices 358 may include a presence-sensitive screen, a touch screen, a gesture responsive system, a mouse, a keyboard, a voice responsive system, video camera, microphone or any other type of device for detecting a command from a user.

In some embodiments, one or more output devices 360 may also be included in mobile communications device or computing device 300. In embodiments, output device 360, in some examples, may be configured to provide output to a user using tactile, audio, or video output. In some embodiments, output devices 360, in one example, may include a presence-sensitive screen or a touch screen. In embodiments, output devices 360 may utilize a sound card, a video graphics adapter card, or any other type of device for converting a signal into an appropriate form understandable to humans or machines. In embodiments, additional examples of output devices 360 may include a speaker, a cathode ray tube (CRT) monitor, a liquid crystal display (LCD), or any other type of device that can generate intelligible output to a user. In some embodiments, an output device 360 may comprise a sound card, one or more sound processing or amplification circuits or modules, and/or one or more speaker assemblies.

In some embodiments, mobile communications device or computing device 300, in some examples, may include one or more power sources or power supplies 362, which may be rechargeable and provide power to mobile communications device or computing device 300. In embodiments, the one or more power sources or power supplies 362 may be one or more rechargeable batteries. In embodiments, the one or more batteries may be made from nickel-cadmium, lithium-ion, or any other suitable material. In another example, in embodiments, the one or more power sources or power supplies 362 may include a power supply connection that receives power from a power source external to mobile communications device or computing device 300.

In some embodiments, one or more application software packages or modules may provide mobile communications device or computing device 300 with additional functionality. In some embodiments, an application software package may include PHI data transfer application software 366. In some embodiments, other software applications may be stored and executed on the mobile communication device which include additional functionality, for example, web browsing, calendar, contacts, games, document processing, or any other functionality performed by a computing device such as mobile communications device or computing device 300. In some embodiments, one or more software application packages (or device drivers) may cause at least one of output devices 360 to display some elements of a graphical user interface (GUI). In embodiments, mobile communications device or computing device 300 may include one or more operating systems 364. In some embodiments, operating systems 364, in some examples, may control the operation of components of mobile communications device or computing device 300. For example, operating system 364, in one example, may facilitate the interaction of other software applications with the processors 350, memory device 352, communication or network interfaces 354, storage devices 356, input devices 358, output devices 360, and power sources or power supplies 362.

In some embodiments, at least one of the other application software packages may be a part of one or more operating systems 364 operating and being executed by one or more processors 350 of a mobile communications device or computing device 300. In some examples, in embodiments, at least one of the other application software may receive input from one or more input devices 358 of mobile communications device or computing device 300. In embodiments, the at least one of the other application software packages may, for example, receive at least one of audio or video information associated with a communication session from other computing devices participating in the communication session.

In some embodiments, a method for transferring patient-related data stored on a smartphone to diagnostic medical equipment utilizing near-field communications (NFC) includes downloading a protected health information (PHI) transfer software application onto the smartphone, the PHI transfer software application being part of a larger medical provider software application that may comprise patient appointment scheduling functionality and/or medication management functionality; initiating search for NFC-capable devices in adjacent areas to the smartphone; establishing a communication session between the smartphone and any found NFC-capable device; initiating data transfer protocol and transferring patient's PHI from smartphone to the found NFC-capable device; and closing the communication session and the PHI transfer software application. In some embodiments, the method for transferring patient-related data stored on a smartphone further includes verifying an accuracy of a patient's PHI before initiating the data transfer protocol.

In some embodiments, a mobile communication device includes one or more processors; one or more memory devices; one or more NFC communication modules; computer-readable instructions stored in the one or more memory devices and executable by the one or more processors, the computer-readable instructions, when executable by the one or more processors, to cause the mobile communication device to: (a) initiate a search for an NFC-capable medical diagnostic device in adjacent areas to the mobile communication device; (b) receive one or more messages from an established NFC-capable medical diagnostic device identifying that the established NFC-capable medical diagnostic device is able to communicate with the mobile communication device; (c) initiate a communication session between the mobile communication device and the established NFC-capable medical diagnostic device; and (d) initiate a transfer of a patient's PHI from a mobile communication device to the established NFC-capable medical diagnostic device utilizing the NFC protocol. In some embodiments, computer-readable instructions, when executable by the one or more processors, further to cause the mobile communication device to: terminate the communication session between the mobile communication device and the established NFC-capable medical diagnostic device; and close a PHI transfer software application on the mobile communication device. In some embodiments, the computer-readable instructions, when executable by the one or more processors, further cause the mobile communication device to download the PHI transfer software application to the mobile communication device before initiating the search for the NFC-capable medical diagnostic device. In some embodiments, the computer-readable instructions, when executable by the one or more processors, further to download a medical provider software application to the mobile communication device, wherein the medical provider software application includes a PHI transfer software application. In some embodiments, the mobile communication device includes a display and wherein the computer-readable instructions, when executable by the one or more processors, further causes the mobile communication device to communicate the PHI data to be transferred to the display of the mobile communication device to verify that the PHI data to be transferred to the established NFC-capable medical diagnostic device is the correct PHI data. In some embodiments, the NFC communications module is attached or connected to a housing of the mobile communication device.

In some embodiments, a medical diagnostic device includes one or more processors; one or more memory devices; one or more NFC communication modules; computer-readable instructions stored in the one or more memory devices and executable by the one or more processors, the computer-readable instructions, when executable by the one or more processors, to cause the mobile communication device to: (a) receive one or more messages from an NFC-capable mobile communication device identifying that the NFC-capable mobile communication device is able to communicate with the medical diagnostic device; (b) establish a communication session between the one or more NFC communication modules of the medical diagnostic device and the NFC-capable mobile communication device; and (c) receive patient's personal health information (PHI) from the mobile communication device via the NFC communication module; and utilize the patient's PHI to register the patient with the medical diagnostic device. In some embodiments, the computer-readable instructions are further executable by the one or more processors to store the patient's PHI in the one or more memory devices of the medical diagnostic device. In some embodiments, the computer-readable instructions are further executable by the one or more processors to associate the patient's PHI with a medical record already stored in the one or more memory devices of the medical diagnostic device. In some embodiments, the computer-readable instructions are further executable by the one or more processors to initiate communication with the NFC-capable mobile communication device to initially locate the mobile communication device. In some embodiments, the medical diagnostic device further includes a display screen, the computer-readable instructions are further executable by the one or more processors to communicate the received patient PHI information to the display screen to allow the patient to verify the received PHI information. In some embodiments, the one or more processors, the one or more memory devices, the computer-readable instructions and the NFC communications module are physically located in a computing device that is coupled to the medical diagnostic device. In some embodiments, the NFC communication module is part of an accessory device connected or attached to a housing of the medical diagnostic device. In some embodiments, a PHI transfer software application is downloaded to the one or more memory devices before communication with the NFC-enabled mobile communication device. In some embodiments, the communication session between the medical diagnostic device and the NFC-enable mobile communication device may be encrypted.

For the purposes of this disclosure, a system or module may be software, hardware, or firmware (or combinations thereof), process or functionality, or component thereof, that performs or facilitates the processes, features, and/or functions described herein (with or without human interaction or augmentation). In embodiments, a chipset may include hardware, software stored in memory, and/or firmware. In embodiments, a module can include sub-modules, subroutines, components, subcomponents. In embodiments, software components and/or subcomponents of a module may be stored on a computer readable medium and/or memory device. In embodiments, systems and/or modules may be integral to and/or installed one or more computing devices (e.g., application servers, mobile application servers, and/or cloud-based servers) may be loaded and executed by one or more processors on one or more computing device. In embodiments, one or more software systems and/or modules may be grouped into an engine and/or an application.

Those skilled in the art may recognize that the methods, apparatus and systems of the present disclosure may be implemented in many manners and, as such are not to be limited by the foregoing exemplary embodiments and/or examples. In other words, functional elements being performed by single or multiple components, in various combinations of hardware and software or firmware, and individual functions, may be distributed among software applications at either the client or server, or multiple computing devices, or both. In this regard, any number of the features of the different embodiments described herein may be combined into single or multiple embodiments, and alternate embodiments having fewer than, or more than, all of the features described herein are possible. Functionality may also be, in whole or in part, distributed among multiple components and/or assemblies, in manners now known or to become known. Thus, myriad software/hardware/firmware combinations may be possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

In embodiments, communications between a mobile communications device and/or a computing device and a wireless network may be in accordance with known and/or to be developed communication network protocols including, for example, global system for mobile communications (GSM), enhanced data rate for GSM evolution (EDGE), 802.11b/g/n, and/or worldwide interoperability for microwave access (WiMAX). In embodiments, a mobile communications/computing device and/or a networking device may also have a subscriber identity module (SIM) card, which, for example, may comprise a detachable smart card that is able to store subscription content of a user, and/or is also able to store a contact list of the user. In embodiments, a user may own a mobile communications/computing device and/or networking device or may otherwise be a user, such as a primary user, for example. In embodiments, a mobile communications device may be assigned an address by a wireless network operator, a wired network operator, and/or an Internet Service Provider (ISP). For example, an address may comprise a domestic or international telephone number, an Internet Protocol (IP) address, MAC address and/or one or more other identifiers. In other embodiments, a communication network may be embodied as a wired network, wireless network, or any combinations thereof. In embodiments, communications between a mobile computing device or computing device and/or a network device and a wireless network may be in accordance with known and/or to be developed communication network protocols including, for example, global system for mobile communications (GSM), enhanced data rate for GSM evolution (EDGE), 802.11b/g/n, and/or worldwide interoperability for microwave access (WiMAX). In other embodiments, a communication network may be embodied as a wired network, wireless network, or any combinations thereof.

Algorithmic descriptions and/or symbolic representations are examples of techniques used by those of ordinary skill in the signal processing and/or related arts to convey the substance of their work to others skilled in the art. An algorithm here, and generally, is considered to be a self-consistent sequence of operations and/or similar signal processing leading to a desired result. In this context, operations and/or processing involve physical manipulation of physical quantities. Typically, although not necessarily, such quantities may take the form of electrical and/or magnetic signals and/or states capable of being stored, transferred, combined, compared, processed or otherwise manipulated as electronic signals and/or states representing various forms of content, such as signal measurements, text, images, video, audio, etc. It has proven convenient at times, principally for reasons of common usage, to refer to such physical signals and/or physical states as bits, values, elements, symbols, characters, terms, numbers, numerals, measurements, content and/or the like. It should be understood, however, that all of these and/or similar terms are to be associated with appropriate physical quantities and are merely convenient labels. Unless specifically stated otherwise, as apparent from the preceding discussion, it is appreciated that throughout this specification discussions utilizing terms such as “processing,” “computing,” “calculating,” “determining”, “establishing”, “obtaining”, “identifying”, “selecting”, “generating”, and/or the like may refer to actions and/or processes of a specific apparatus, such as a special purpose computer and/or a similar special purpose computing and/or network device. In the context of this specification, therefore, a special purpose computer and/or a similar special purpose computing and/or network device is capable of processing, manipulating and/or transforming signals and/or states, typically represented as physical electronic and/or magnetic quantities within memories, registers, and/or other storage devices, transmission devices, and/or display devices of the special purpose computer and/or similar special purpose computing and/or network device. In the context of this particular patent application, as mentioned, the term “specific apparatus” may include a general-purpose computing and/or network device, such as a general-purpose computer, once it is programmed to perform particular functions pursuant to instructions from program software.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

As detailed above, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor. The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of.” Finally, for ease of use, the terms “including,” “incorporating,” “includes,” “incorporates,” and “having” (and their derivatives), as used in the specification and claims, are interchangeable with and shall have the same meaning as the word “comprising.”

While certain exemplary techniques have been described and shown herein using various methods and systems, it should be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular examples disclosed, but that such claimed subject matter may also include all implementations falling within the scope of the appended claims, and equivalents thereof.

1. A method for transferring patient-related data stored on a smartphone to diagnostic medical equipment utilizing near-field communications (NFC), comprising:
downloading a protected health information (PHI) transfer software application onto the smartphone, the PHI transfer software application being part of a larger medical provider software application that may comprise patient appointment scheduling functionality and/or medication management functionality;
initiating search for NFC-capable devices in adjacent areas to the smartphone;
establishing a communication session between the smartphone and any found NFC-capable device;
initiating data transfer protocol and transferring patient's PHI from smartphone to the found NFC-capable device; and
closing the communication session and the PHI transfer software application.

2. The method of claim 1, further comprising verifying an accuracy of a patient's PHI before initiating the data transfer protocol.

3. A mobile communication device, comprising:
one or more processors;
one or more memory devices;
one or more NFC communication modules;
computer-readable instructions stored in the one or more memory devices and executable by the one or more processors, the computer-readable instructions, when executable by the one or more processors, to cause the mobile communication device to:
initiate a search for an NFC-capable medical diagnostic device in adjacent areas to the mobile communication device;
receive one or more messages from an established NFC-capable medical diagnostic device identifying that the established NFC-capable medical diagnostic device is able to communicate with the mobile communication device;
initiate a communication session between the mobile communication device and the established NFC-capable medical diagnostic device; and
initiate a transfer of a patient's PHI from a mobile communication device to the established NFC-capable medical diagnostic device utilizing the NFC protocol.

4. The mobile communication device of claim 3, the computer-readable instructions, when executable by the one or more processors, further to cause the mobile communication device to:
terminate the communication session between the mobile communication device and the established NFC-capable medical diagnostic device; and
close a PHI transfer software application on the mobile communication device.

5. The mobile communication device of claim 4, the computer-readable instructions, when executable by the one or more processors, further to download the PHI transfer software application to the mobile communication device before initiating the search for the NFC-capable medical diagnostic device.

6. The mobile communication device of claim 3, the computer-readable instructions, when executable by the one or more processors further to download a medical provider software application to the mobile communication device, wherein the medical provider software application includes a PHI transfer software application.

7. The mobile communication device of claim 3, further comprising a display and wherein the computer-readable instructions, when executable by the one or more processors, further to communicate the PHI data to be transferred to the display of the mobile communication device to verify that the PHI data to be transferred to the established NFC-capable medical diagnostic device is the correct PHI data.

8. The mobile communication device of claim 3, wherein the NFC communications module is attached or connected to a housing of the mobile communication device.

9. A medical diagnostic device, comprising:
one or more processors;
one or more memory devices;
one or more NFC communication modules;
computer-readable instructions stored in the one or more memory devices and executable by the one or more processors, the computer-readable instructions, when executable by the one or more processors, to cause the mobile communication device to:
receive one or more messages from an NFC-capable mobile communication device identifying that the NFC-capable mobile communication device is able to communicate with the medical diagnostic device;
establish a communication session between the one or more NFC communication modules of the medical diagnostic device and the NFC-capable mobile communication device; and
receive patient's personal health information (PHI) from the mobile communication device via the NFC communication module; and
utilize the patient's PHI to register the patient with the medical diagnostic device.

10. The medical diagnostic device of claim 9, the computer-readable instructions further executable by the one or more processors to store the patient's PHI in the one or more memory devices of the medical diagnostic device.

11. The medical diagnostic device of claim 10, the computer-readable instructions further executable by the one or more processors to associate the patient's PHI with a medical record already stored in the one or more memory devices of the medical diagnostic device.

12. The medical diagnostic device of claim 9, the computer-readable instructions further executable by the one or more processors to initiate communication with the NFC-capable mobile communication device to initially locate the mobile communication device.

13. The medical diagnostic device of claim 9, further comprising a display screen, the computer-readable instructions further executable by the one or more processors to communicate the received patient PHI information to the display screen to allow the patient to verify the received PHI information.

14. The medical diagnostic device of claim 9, wherein the one or more processors, the one or more memory devices, the computer-readable instructions and the NFC communications module are physically located in a computing device that is coupled to the medical diagnostic device.

15. The medical diagnostic device of claim 9, wherein the NFC communication module is part of an accessory device connected or attached to a housing of the medical diagnostic device.

16. The medical diagnostic device of claim 9, wherein a PHI transfer software application is downloaded to the one or more memory devices before communication with the NFC-enabled mobile communication device.

17. The mobile communication device of claim 3, wherein the communication session between the mobile communication device and the NFC-capable diagnostic device is encrypted.

18. The medical diagnostic device of claim 9, wherein the communication session between the medical diagnostic device and the NFC-enabled mobile communication device is encrypted.
